(19) United States
(12) Patent Application Publication
(10) Pub. No.: US 2021/0254001 A1
(43) Pub. Date: Aug. 19, 2021

(54) FcyRIIIa signaling generates Bcl6+PD1+CD4+ Tfh-like cells

(71) Applicant: Anil Kumar Chauhan, Wildwood, MO (US)

(72) Inventor: Anil Kumar Chauhan, Wildwood, MO (US)

(21) Appl. No.: 16/792,204

(22) Filed: Feb. 15, 2020

(51) Int. Cl. C12N 5/0783

(52) U.S. Cl. CPC C12N 5/0636; C12N 2506/11

(57) ABSTRACT

Cosignaling by immune complexes in human CD4⁺ T cells triggers co-expression of low affinity Fc receptors, which upon signaling trigger Bcl6, PD1, IFN-γ, IL-17A, and IL-21 expression. A small molecular chemical entity, a protein-biologics, peptide that inhibits production/expansion of these cells to obtain beneficial outcome in treating autoimmunity and viral infections. Quantitation of the cells that express Fc receptors in combination to PD1, Bcl6, IFN-γ, IL17A, IL-21 for the assessment of the disease activity in autoimmunity and viral infections.

### DESCRIPTION OF INVENTION AND BACKGROUND

Antigens present in the circulation or expressed on the cell surface bind to the antibodies directed to these free antigens/or antigens expressed and retained on membrane by cells, and form what are described as antigen-antibody or immune complexes (ICs). These complexes show heterogeneity in the molecular size, antigen composition, complement fragment opsonization and trigger effector functions in lymphocytes. We earlier reported the composition of purified ICs using both 2D SDS-PAGE analysis and nano-LC/MS/MS from autoimmune diseases and malignancies (Chauhan and Moore, 2006; Low et al., 2009; Ohyama et al., 2017). ICs bind to low affinity Fc receptors (FcRs) expressed by innate immune cells. We and others have recently, now shown the expression of the low affinity FcRs by a subset of human CD4⁺ T cells (Abdel-Mohsen et al., 2018; Chauhan, 2016, 2017b; Chauhan et al., 2015; Holgado et al., 2018). We have further established that the FcR signaling is capable of generating CD4⁺ T effector populations and trigger the upregulation as well as relocate the nucleic-acid sensing toll-like receptors (NA-TLRs) to the cell surface (Chauhan, 2017a; Chauhan et al., 2015; Chauhan et al., 2016).

Human CD4⁺ T cells are critical to the host's immune defense. Aberrant signaling events in the CD4⁺ T cells, result in the development of autoimmunity and/or other immune mediated injuries. Based on the type of cytokines produced by these cells and transcription factor expression, five major populations of effector CD4⁺ T cells, T_{H}1, T_{H}2, T_{H}17, Treg (regulatory) and Tfh (follicular helper) are defined. These populations show plasticity and are target for new therapeutic interventions (DuPage and Bluestone, 2016). In certain human subjects, the ICs formation by the biological drugs trigger adverse immune reactions. We have reported a direct role of these ICs in altering the CD4⁺ T cell responses and triggering the cytokine production (Chauhan et al., 2015; Chauhan et al., 2016).

Human naïve CD4⁺ T cells entering the periphery, continues to expand and differentiate. The fate of these cells in the periphery is further defined by the signals they receive from the antigen presenting cells (APC) and the local environment i.e. cytokine milieu and surrounding tissue antigens. In the periphery a productive signaling by T cell receptor complex (TCR) from peptide-MHC binding and co-stimulation from CD28 is needed for cellular activation. TCR engagement in the absence of CD28 results in an unresponsive anergic state.

Earlier studies have demonstrated the expression of FcRs in small populations of CD4⁺ T cells, however what role these receptors play in T cell mediated immunity was not defined and recognized (Sandor and Lynch, 1993). It was suggested that the FcRs participate in the suppression and activation of T cells (Sandor and Lynch, 1993). Whether T cells express FcRs remain a long-lasting controversy. The conventional wisdom suggests that T cells do not express FcRs (Bournazos and Ravetch, 2017; Bruhns and Jonsson, 2015; Nimmerjahn and Ravetch, 2008). This concept was based on unsupported comments made in these review articles and no experimental data was provided or exists to support this claim (Nimmerjahn and Ravetch, 2008). In 2011, we first reported that the FcRs ligation by ICs in CD4⁺ T Jurkat cell line and purified human naïve CD4⁺ T cells, phosphorylate TCR complex proteins (Chauhan and Moore, 2011). Thereafter, we also established a role for this signaling in the generation of the effector CD4⁺ T cell subsets (Chauhan et al., 2015; Chauhan et al., 2016). Now several other groups have accepted/reported the presence of Fc receptors and proposed functional roles for these receptors in CD4⁺ T cell responses (Abdel-Mohsen et al., 2018; Holgado et al., 2018; Martin et al., 2018; Rasoulouniriana et al., 2019). Subsequently, we have also shown that this signaling via Fc receptor (particularly CD16a or FcγRIIIa) upregulates NA-TLRs in CD4⁺ T cells and the synergistic signaling via these receptors upregulates key pro inflammatory cytokine production (Chauhan, 2017a; Chauhan et al., 2016). I have summarized this historical perspective in a recent review article (Chauhan, 2016).

The FcRs, besides promoting and regulating the inflammatory responses, also play a critical role in regulating the B cell responses by modulating the threshold of the B cell receptor (BCR). FcRs ligation by ICs, in addition to the activation of immune effector cells, by cross-linking the low affinity FcγRIIB with B cell receptor (BCR) also regulates the threshold of B cell activation; hence, regulating the antibody production (Smith and Clatworthy, 2010). These receptors besides utilizing calcium dependent pathways also trigger the signaling via the RAS-RAF-MAPK. A co-synergistic role for BCR and nucleic acid sensing toll-like receptors (NA-TLRs) have been proposed (Rawlings et al., 2017). We have reported a synergistic role of FcRs with NA-TLRs in CD4⁺ T cells (Chauhan, 2017a, and FIG. 5).

Recently, several groups have disclosed the expression of CD32a (FcγRIIa) by CD4⁺ T cells. These CD32a⁺ expressing cells exhibit enrichment of HIV-1 provirus (Abdel-Mohsen et al., 2018; Descours et al., 2017; Martin et al., 2018). Similar to our observation of activation induced expression of CD16a, another group showed the expression of CD32a in activated CD4⁺ T cells (Holgado et al., 2018). Now a report has also suggested the role for μ-chain receptors in the activation of CD4⁺ T cells (Meryk et al., 2019). A role for high affinity FcγRI expression on CD4+Th1 cells in antibody mediated cytotoxic activity is also now suggested (Rasoulouniriana et al., 2019). The authors of this study also highlighted the controversy on the FcRs expression on CD4⁺ T cells and cited our work as the new emerging paradigm in their discussion (Rasoulouniriana et al., 2019). We showed that both in SLE and HIV-1 infected cells FcR expressing population is expanded (FIG. 1). Several studies that have recognized the role of CD32a in HIV-1 provirus enrichment and emphasized the controversy on the expression of FcRs in CD4⁺ T cell population (Abdel-Mohsen et al., 2018; Martin et al., 2018). Strangely, we have also observed the overexpression of CCR5, a GPCR protein that act as a coreceptor for HIV (FIG. 7). Cumulative, these studies and our work now provides a solid evidence for a demonstrated role for Fc receptor signaling in CD4⁺ T cell function.

### Fc Receptors Signaling Participate in the Expression of T Cell Regulatory Proteins

Our work has established the presence of a new CD4⁺ FcγRIIIa⁺ population that produces high levels of IFN-γ upon ICs engagement (Chauhan et al., 2015). Similar results were also reported for an another receptor of this family FcγRIIa (CD32a) (Holgado et al., 2018). Ligation of these receptors by ICs triggers robust IFN-γ production (Chauhan et al., 2015; Holgado et al., 2018; Rasoulouniriana et al., 2019). IFN-γ augments the antigen processing, cytokine and chemokine production required for the myeloid cell recruitment to the site of inflammation. In addition, IFN-γ is also required for the expression of toll-like receptors (TLRs), nitric oxide synthase, and phagocyte oxidase by macrophages (Hu and Ivashkiv, 2009). Thus, in the disease pathology, FcγR⁺IFN-γ⁺ T_{H} subset observed by us will contribute to the altered ratio of T_{H}1/T_{H}17 cells and this will contribute to the disease pathology (Chauhan et al., 2015; Chauhan et al., 2016). We examined and reported the production of IFN-γ and IL-17 cytokines in response to FcRs activation in human naïve CD4⁺ T cells (Chauhan et al., 2015; Chauhan et al., 2016). Upon Fc-receptor signaling CD4⁺ T cells that bind to ICs show pSyk, express Bcl6, and produce IFN-y (FIG. 2). These pSyk⁺ cells express Tfh population markers such as ICOS, CXCR5 and PD1. The PD1^{int} cells that express intermediate amount of PD1 protein are those cells that express Bcl6 along with producing IFN-γ (FIG. 3). Fc signaling in vitro generated these cells (FIG. 4). We have also show that such cells produce IL-21 and inhibiting Syk phosphorylation blocks IL-21 production (FIG. 4). FcR signaling show synergy with toll-like receptor 9 (TLR9) signaling from oligonucleotide CpG ODN 2006 in enhancing the production of IL-17 and IL-21 in cells that also expressed Bcl6 (FIG. 5). RNA-seq expression analysis upon FcR signaling also showed enhanced expression of Proteasome complex, MHC class II, TCR signaling and NF-κB signaling transcripts (FIG. 6). Granzyme A (GZMA) and chemokine receptor 5 (CCR5) were also upregulated (FIG. 7). Upon co-culturing of these Tfh cells with human naïve B cells triggered production of IgD, IgM, and expression of CD38 and CD27 suggesting differentiation into plasma B cells (FIG. 8).

The T_{H}1 memory effector T cells produce high levels of IFN-γ (O'Shea and Paul, 2010). IFN-γ is an important mediator of immunity and inflammation. IFN-γ can either augment or suppress autoimmunity and the associated pathology in a disease specific manner (Hu and Ivashkiv, 2009). The T_{H}1 subset, which in addition to producing IFN-γ, also express transcription factor T-bet exclusively, and protects the host against the intracellular infections including viruses and Toxoplasma (Abbas et al., 1996; Agnello et al., 2003; Aliberti et al., 2004). T_{H}1 cells contribute to the development of autoimmunity due to a wide array of functions associated with IFN-γ. The T_{H}17, another subset of effector memory cells subset that is associated with proinflammatory responses along with T_{H}1 and these subsets contribute to the pathology in a number of autoimmune diseases (Annunziato et al., 2009; Kyttaris et al., 2010; Saijo et al., 2010; Steinman, 2010). The antigen specific T_{H}17 cells once polarized, demonstrate a loss of IL-17A production with acquisition of the IFN-γ production (Lee et al., 2009). While T_{H}1 initiate tissue damage, T_{H}17 in addition to causing the damage, also sustains tissue damage in immune mediated injuries during organ-specific autoimmunity in the synovium, heart, skin, infections and brain. ICs stimulation of human naïve CD4⁺ T cells triggers the development of both T_{H}1 and T_{H}17 cells and this results in the upregulation of interferon signature gene expression profile (Chauhan et al., 2016).

The costimulatory inhibitors on T lymphocytes i.e. cytotoxic T lymphocyte antigen-4 (CTLA-4) and programmed death-1 (PD1) have been successfully drugged (Fife and Bluestone, 2008). The PD1 provides an inhibitory signal to the immune response and its inhibition with antibodies have been used successfully to treat cancers (LaFleur et al., 2018; Sharpe and Pauken, 2017). PD1 signaling has multiple role in regulating the autoreactive T cells. Autoreactive T cells in the target tissue express high levels of PD1 (Mueller et al., 2010). We and others have shown the presence of PD1 protein in CD4⁺ T cells (Chauhan et al., 2016; Sharpe and Pauken, 2017). Repeated activation of T cells via TCR signaling triggers expression of PD1. Sustained expression of PD1 on T cells drives these cells into dysfunctional state “exhaustion” (Barber et al., 2006). It has been shown that PD1 constrains self-reactive CD4⁺ and CD8⁺ effector T cells. A role for PD1 signaling in regulating chronic lymphocytic choriomeningitis (LCMV), HIV, hepatitis C, hepatitis B is also suggested. During viral infections, T cells lose their ability to produce IL-2, TNF-alpha, IFN-γ and enters the state of exhaustion (Wherry et al., 2003). It is now proposed that T cells with intermediate expression of PD1 levels are dysfunctional, but can be reinvigorated, whereas cells with high levels of PD1 expression are terminally exhausted (Sen et al., 2016). Our work showed that the cells with moderate (intermediate) PD1 expression that are FcγRIIIa⁺ from challenge with ICs, trigger IFN-γ related mRNAs profile (Chauhan et al., 2016). Such a response predicts clinical response to PD1 blockage (Ayers et al., 2017). Most patients receiving anti-PD1 therapy do not show long lasting remission and many are refractory to immune-check point blockade therapies (LaFleur et al., 2018; Sharpe and Pauken, 2017). PD1 is expressed by Tfh cells, and this expression is governed by transcriptional regulator B cell lymphoma 6 (Bcl6) protein.

Tfh cells support the differentiation of antigen-specific B cells into memory and plasma B cells (CD27⁺CD38⁺). The CD4⁺ T cells that express high levels of CXCR5, PD1 and inducible T cell costimulator (ICOS), IL-21, and transcriptional regulator Bcl6 protein are considered to be Tfh subset. Expression of Bcl6 in human circulating (peripheral) Tfh-like cells is controversial. Although, in lupus peripheral Tfh-like cells express Bcl6, the Bcl6 is shown to be absent in such cells in HIV-1 infected patients (Tangye et al., 2013). Tfh cells in human germinal centers express high levels of CXCR5 (a GPCR signaling protein), PD1 and ICOS (Ueno et al., 2015). Tfh precursors in tonsils appear to lack Bcl6 expression (Campbell et al., 2001). The molecular mechanisms by which peripheral blood Tfh maintain their Tfh characteristics remains largely unknown (Ueno et al., 2015). ICOS expression increases after vaccination, otherwise limited to small population of cells. Influenza vaccination transiently induces ICOS exclusively on blood memory cells (Bentebibel et al., 2013). Generation and activation of Tfh cells contributes to the pathogenesis of autoimmune diseases. IFN-γ signaling blockade alleviate Tfh and GC B cells accumulation and clinical manifestation of Roquin^{san} mouse model (Linterman et al., 2009). Tfh cells in MRL/lpr (SLE model) share features of GC Tfh cells and their development is dependent on ICOS and Bcl6 and their helper function is dependent on IL-21 (Odegard et al., 2008). In this invention, we show a role for FcRs signaling in the expression of ICOS and PD1 expression in human CD4⁺ T cells (FIGS. 3,4). IC signaling in addition to the generation of PD1 cells, trigger production of IFN-γ, IL-17A, IL-21 cytokines and expression of Bcl6. Those cells that express high levels of PD1 (marked as PD1^{high}) do not show these properties.

Strangely enough, now several groups have implicated Fc mediated responses and the formation of ICs in the beneficial outcome observed during HIV-1 vaccination observed in trials such as RV 144 (Grobben et al., 2019). A functional role for FcR signaling has also been established in the undergoing trials for HIV-1 vaccination using broadly neutralizing antibodies (Parsons et al., 2018). Thus far these reports have not studied the role for FcR expression on CD4⁺ T cells. Tfh cells show enrichment of HIV-1 and is the most infected subset of CD4⁺ T cells (Pallikkuth et al., 2015). We have established that the FcR signaling contribute to the development of Tfh-like cells. Another important role for y-chain in CD4 T cells has shown to be the induction of antibody dependent cellular mediated cytotoxicity (ADCC) (O'llier et al., 2017). Our experiments showed induced expression of granzyme A (GZMA), a serine protease utilized by cytotoxic T cells to kill target cells (drawing no. 7).

Modulation of FcR signaling in CD4⁺ T cells leading to alteration in immune check point responses using a peptide analog, therapeutic antibody, small chemical entities, or any biological and chemical means will provide a useful mode of treatment of many autoimmune diseases and viral infections. Such modulations will be useful in those diseases where elevated levels of ICs are observed and by activating complement pathway contribute to tissue/organ damage.

### LITERATURE CITED

Abbas, A. K., Murphy, K. M., and Sher, A. (1996). Functional diversity of helper T lymphocytes. Nature 383, 787-793.
Abdel-Mohsen, M., Kuri-Cervantes, L., Grau-Exposito, J., Spivak, A. M., Nell, R. A., Tomescu, C., Vadrevu, S. K., Giron, L. B., Serra-Peinado, C., Genesca, M., et al. (2018). CD32 is expressed on cells with transcriptionally active HIV but does not enrich for HIV DNA in resting T cells. Sci Transl Med 10.
Agnello, D., Lankford, C. S., Bream, J., Morinobu, A., Gadina, M., O'Shea, J. J., and Frucht, D. M. (2003). Cytokines and transcription factors that regulate T helper cell differentiation: new players and new insights. J Clin Immunol 23, 147-161.
Aliberti, J., Jankovic, D., and Sher, A. (2004). Turning it on and off: regulation of dendritic cell function in Toxoplasma gondii infection. Immunol Rev 201, 26-34.
Annunziato, F., Cosmi, L., Liotta, F., Maggi, E., and Romagnani, S. (2009). Type 17 T helper cells-origins, features and possible roles in rheumatic disease. Nat Rev Rheumatol 5, 325-331.
Ayers, M., Lunceford, J., Nebozhyn, M., Murphy, E., Loboda, A., Kaufman, D. R., Albright, A., Cheng, J. D., Kang, S. P., Shankaran, V., et al. (2017). IFN-gamma-related mRNA profile predicts clinical response to PD-1 blockade. The Journal of clinical investigation 127, 2930-2940.
Barber, D. L., Wherry, E. J., Masopust, D., Zhu, B., Allison, J. P., Sharpe, A. H., Freeman, G. J., and Ahmed, R. (2006). Restoring function in exhausted CD8 T cells during chronic viral infection. Nature 439, 682-687.
Bentebibel, S. E., Lopez, S., Obermoser, G., Schmitt, N., Mueller, C., Harrod, C., Flano, E., Mejias, A., Albrecht, R. A., Blankenship, D., et al. (2013). Induction of ICOS+CXCR3+CXCR5+T_{H} cells correlates with antibody responses to influenza vaccination. Sci Transl Med 5, 176ra132.
Bournazos, S., and Ravetch, J. V. (2017). Fcgamma Receptor Function and the Design of Vaccination Strategies. Immunity 47, 224-233.
Bruhns, P., and Jonsson, F. (2015). Mouse and human FcR effector functions. Immunol Rev 268, 25-51.
Campbell, D. J., Kim, C. H., and Butcher, E. C. (2001). Separable effector T cell populations specialized for B cell help or tissue inflammation. Nature immunology 2, 876-881.
Chauhan, A. K. (2016). Human CD4⁺ T-cells-A Role for low affinity Fc-receptors. Front Immunol 10.3389/fimmu.2016.00215.
Chauhan, A. K. (2017a). FcgammaRIIIa Signaling Modulates Endosomal TLR Responses in Human CD4+ T Cells. J Immunol 198, 4596-4606.
Chauhan, A. K. (2017b). Immune complexes in disease pathology. Front. Immunol. 8, 173.
Chauhan, A. K., Chen, C., Moore, T. L., and DiPaolo, R. J. (2015). Induced Expression of FcgammaRIIIa (CD16a) on CD4⁺ T Cells Triggers Generation of IFN-gammahigh Subset. The Journal of biological chemistry 290, 5127-5140.
Chauhan, A. K., and Moore, T. L. (2006). Presence of plasma complement regulatory proteins clusterin (Apo J) and vitronectin (S40) on circulating immune complexes (CIC). Clinical and experimental immunology 145, 398-406.
Chauhan, A. K., and Moore, T. L. (2011). T cell activation by terminal complex of complement and immune complexes. The Journal of biological chemistry 286, 38627-38637.
Chauhan, A. K., Moore, T. L., Bi, Y., and Chen, C. (2016). FcgammaRIIIa-Syk Co-signal Modulates CD4⁺ T-cell Response and Up-regulates Toll-like Receptor (TLR) Expression. The Journal of biological chemistry 291, 1368-1386.
Descours, B., Petitjean, G., Lopez-Zaragoza, J. L., Bruel, T., Raffel, R., Psomas, C., Reynes, J., Lacabaratz, C., Levy, Y., Schwartz, O., et al. (2017). CD32a is a marker of a CD4 T-cell HIV reservoir harbouring replication-competent proviruses. Nature 543, 564-567.
DuPage, M., and Bluestone, J. A. (2016). Harnessing the plasticity of CD4(+) T cells to treat immune-mediated disease. Nature reviews. Immunology 16, 149-163.
Fife, B. T., and Bluestone, J. A. (2008). Control of peripheral T-cell tolerance and autoimmunity via the CTLA-4 and PD-1 pathways. Immunol Rev 224, 166-182.
Grobben, M., Stuart, R. A., and van Gils, M. J. (2019). The potential of engineered antibodies for HIV-1 therapy and cure. Curr Opin Virol 38, 70-80.
Holgado, M. P., Sananez, I., Raiden, S., Geffner, J. R., and Arruvito, L. (2018). CD32 Ligation Promotes the Activation of CD4(+) T Cells. Front Immunol 9, 2814.
Hu, X., and Ivashkiv, L. B. (2009). Cross-regulation of signaling pathways by interferon-gamma: implications for immune responses and autoimmune diseases. Immunity 31, 539-550.
Kyttaris, V. C., Zhang, Z., Kuchroo, V. K., Oukka, M., and Tsokos, G. C. (2010). Cutting edge: IL-23 receptor deficiency prevents the development of lupus nephritis in C57BL/6-lpr/lpr mice. J Immunol 184, 4605-4609.
LaFleur, M. W., Muroyama, Y., Drake, C. G., and Sharpe, A. H. (2018). Inhibitors of the PD-1 Pathway in Tumor Therapy. J Immunol 200, 375-383.
Lee, Y. K., Turner, H., Maynard, C. L., Oliver, J. R., Chen, D., Elson, C. O., and Weaver, C. T. (2009). Late developmental plasticity in the T helper 17 lineage. Immunity 30, 92-107.
Linterman, M. A., Rigby, R. J., Wong, R. K., Yu, D., Brink, R., Cannons, J. L., Schwartzberg, P. L., Cook, M. C., Walters, G. D., and Vinuesa, C. G. (2009). Follicular helper T cells are required for systemic autoimmunity. The Journal of experimental medicine 206, 561-576.
Low, J. M., Chauhan, A. K., Gibson, D. S., Zhu, M., Chen, S., Rooney, M. E., Ombrello, M. J., and Moore, T. L. (2009). Proteomic analysis of circulating immune complexes in juvenile idiopathic arthritis reveals disease-associated proteins. Proteomics. Clinical applications 3, 829-840.
Martin, G. E., Pace, M., Thornhill, J. P., Phetsouphanh, C., Meyerowitz, J., Gossez, M., Brown, H., Olejniczak, N., Lwanga, J., Ramjee, G., et al. (2018). CD32-Expressing CD4 T Cells Are Phenotypically Diverse and Can Contain Proviral HIV DNA. Front Immunol 9, 928.
Meryk, A., Pangrazzi, L., Hagen, M., Hatzmann, F., Jenewein, B., Jakic, B., Hermann-Kleiter, N., Baier, G., Jylhava, J., Hurme, M., et al. (2019). Fcmu receptor as a Costimulatory Molecule for T Cells. Cell Rep 26, 2681-2691 e2685.
Mueller, S. N., Vanguri, V. K., Ha, S. J., West, E. E., Keir, M. E., Glickman, J. N., Sharpe, A. H., and Ahmed, R. (2010). PD-L1 has distinct functions in hematopoietic and nonhematopoietic cells in regulating T cell responses during chronic infection in mice. The Journal of clinical investigation 120, 2508-2515.
Nimmerjahn, F., and Ravetch, J. V. (2008). Fcgamma receptors as regulators of immune responses. Nature reviews. Immunology 8, 34-47.
O'Shea, J. J., and Paul, W. E. (2010). Mechanisms underlying lineage commitment and plasticity of helper CD4⁺ T cells. Science 327, 1098-1102.
O'llier, J., Vivien, R., Vie, H., and Clémenceau, B. (2017). Transfection of FcgRIIIa (CD16) alone can ee sufficient to enable human abTCR T lymphocytes to mediate antibody-dependent cellular cytotoxicity. ImmunoHorizon 1, 63-70.
Odegard, J. M., Marks, B. R., DiPlacido, L. D., Poholek, A. C., Kono, D. H., Dong, C., Flavell, R. A., and Craft, J. (2008). ICOS-dependent extrafollicular helper T cells elicit IgG production via IL-21 in systemic autoimmunity. The Journal of experimental medicine 205, 2873-2886.
Ohyama, K., Yoshimi, H., Aibara, N., Nakamura, Y., Miyata, Y., Sakai, H., Fujita, F., Imaizumi, Y., Chauhan, A. K., Kishikawa, N., and Kuroda, N. (2017). Immune complexome analysis reveals the specific and frequent presence of immune complex antigens in lung cancer patients: A pilot study. Int J Cancer 140, 370-380.
Pallikkuth, S., Sharkey, M., Babic, D. Z., Gupta, S., Stone, G. W., Fischl, M. A., Stevenson, M., and Pahwa, S. (2015). Peripheral T Follicular Helper Cells Are the Major HIV Reservoir within Central Memory CD4 T Cells in Peripheral Blood from Chronically HIV-Infected Individuals on Combination Antiretroviral Therapy. J Virol 90, 2718-2728.
Parsons, M. S., Chung, A. W., and Kent, S. J. (2018). Importance of Fc-mediated functions of anti-HIV-1 broadly neutralizing antibodies. Retrovirology 15, 58.
Rasoulouniriana, D., Santana-Magal, N., Gutwillig, A., Farhat-Younis, L., Wine, Y., Saperia, C., Tal, L., Gutman, H., Tsivian, A., Brenner, R., et al. (2019). A distinct subset of FcgammaRl-expressing Th1 cells exert antibody-mediated cytotoxic activity. The Journal of clinical investigation 129, 4151-4164.
Rawlings, D. J., Metzler, G., Wray-Dutra, M., and Jackson, S. W. (2017). Altered B cell signalling in autoimmunity. Nature reviews. Immunology.
Saijo, S., Ikeda, S., Yamabe, K., Kakuta, S., Ishigame, H., Akitsu, A., Fujikado, N., Kusaka, T., Kubo, S., Chung, S. H., et al. (2010). Dectin-2 recognition of alpha-mannans and induction of Th17 cell differentiation is essential for host defense against Candida albicans. Immunity 32, 681-691.
Sandor, M., and Lynch, R. G. (1993). Lymphocyte Fc receptors: the special case of T cells. Immunology today 14, 227-231.
Sen, D. R., Kaminski, J., Barnitz, R. A., Kurachi, M., Gerdemann, U., Yates, K. B., Tsao, H. W., Godec, J., LaFleur, M. W., Brown, F. D., et al. (2016). The epigenetic landscape of T cell exhaustion. Science 354, 1165-1169.
Sharpe, A. H., and Pauken, K. E. (2017). The diverse functions of the PD1 inhibitory pathway. Nature reviews. Immunology.
Smith, K G., and Clatworthy, M. R. (2010). FcgammaRIIB in autoimmunity and infection: evolutionary and therapeutic implications. Nat Rev Immunol 10, 328-343.
Steinman, L. (2010). Mixed results with modulation of T_{H}-17 cells in human autoimmune diseases. Nat Immunol 11, 41-44.
Tangye, S. G., Ma, C. S., Brink, R., and Deenick, E. K. (2013). The good, the bad and the ugly—TFH cells in human health and disease. Nature reviews. Immunology 13, 412-426.
Ueno, H., Banchereau, J., and Vinuesa, C. G. (2015). Pathophysiology of T follicular helper cells in humans and mice. Nature immunology 16, 142-152.
Wherry, E. J., Blattman, J. N., Murali-Krishna, K., van der Most, R., and Ahmed, R. (2003). Viral persistence alters CD8 T-cell immunodominance and tissue distribution and results in distinct stages of functional impairment. J Virol 77, 4911-4927.

### BRIEF DESCRIPTION OF THE SEVERAL VIEWS OF DRAWINGS

“This patent or application file contains at least five drawing of eight executed in color. Copies of this patent or patent application publication with color drawing(s) will be provided by the Office upon request and the payment of necessary fee”

FIG. 1 establishes the enhanced population of CD4⁺ T cells in SLE and HIV-1 patients. PD1 and Bcl6 (transcription regulator of Tfh cells), Tfh markers are expressed by IC bound cells, which are increased in SLE.

FIG. 1A

Binding of Alexa-488 labeled ICs to CD4⁺ T cells is analyzed using flow cytometry in SLE patient's blood. The FSC-A CD4⁺ gated cells population (A) is enhanced in SLE vs. Control (B). These cells show increased expression of Bcl6 and PD1, known markers for Tfh cells (C).

FIG. 1B

The same type of CD4⁺ T cells as in 1A, analyzed in HIV-1 infected patients untreated, treated with cART and normal control (A). IC binding analyzed in CD8⁺ T cells (B). IC binding analyzed in CD19⁺ B cells (C). IC binding analyzed in CD8^{dim} cells (D).

FIG. 2 establishes that in blood of SLE patients, exists CD4⁺ T cells that binds to ICs and show pSyk. pSyk⁺ cells express Bcl6 and produce IFN-γ.

FIG. 2A

Analysis of SLE patients' blood for CD4⁺ T cells (A) that bound to ICs, show pSyk (B) and pSyk cells express Bcl6 (C)

FIG. 2B

The IC bound CD4⁺ T cells express Bcl6+(A). Bcl6 expressing cells produce IFN-γ (B). Another view of cells that are Bcl6⁺IFN-γ⁺, which are pSyk⁺ (C)

FIG. 2C

pSyk⁺ CD4⁺ T cells are observed as observed as double positive cells pSvk⁺ Bcl6⁺ (r=0.927); pSyk⁺ IFN-γ⁺ (r=0.824) and pSvk⁺ IC⁺ (r=0.864). This establish a role for Syk signaling in the expression of these markers.

FIG. 2D

Bcl6⁺pSyk⁺ double positive cells correlate with Bcl6⁺IFN-γ⁺ cells (r=0.947) and Bcl6+IC⁺ population (r=0.723).

FIG. 3 establishes that pSyk⁺ cells express Tfh markers in the blood. In two populations of PD1⁺ cells seen, one of these population is pSyk⁺ and this population binds to ICs, express Bcl6 and produce IFN-γ.

FIG. 3A

CD4⁺ T cells that are pSyk⁺ express ICOS, CXCR5, PD1 and produce IFN-γ. Two population of PD1 expressing cells are observed, one that is pSyk⁻ and another pSyk⁺ (third from left)

FIG. 3B

pSyk⁺PD1^{int} (marked as intermediate) cells are smaller percentage compared to PD1^{high} pSyk⁻, right Y-axis (p Value=<0.0004) and they also show lower MFI values, left Y-axis (p Value=<0.0064) (A). Cells that are pSyk⁺ and express PD1 does not correlate with total PD1⁺ pSyk⁻ cell numbers (B), A moderate correlation in seen in MFI values in these two cell populations (C).

FIG. 3C

IFN-γ producing cells express PD1 (A). Further analysis show that IFN-γ⁺PD1^{int} cells bind to ICs, and express Bcl6. This is a functional cell population as it produce cytokines.

FIG. 4 establishes that in vitro activation of purified human CD4⁺ T cells with ICs differentiate them into Tfh cells that express Tfh markers, ICOS, PD1, Bcl6, and produce IFN-γ, IL-17A and IL-21. IL-21 production is blocked by inhibiting Syk activation using P505, a Syk inhibitor,

FIG. 4A

Upon in vitro activation by ICs, purified CD4⁺ T cells express ICOS. Treated with ICs (16.4%) vs. untreated control (3.76%).

FIG. 4B

Upon in vitro activation by ICs, purified CD4⁺ T cells express PD1. Treated with ICs (16.0%) vs. untreated control (3.21%)

FIG. 4C

Upon in vitro IC treatment, purified CD4⁺ T cells show PD1⁺ pSyk⁺ cells that correlate with PD1⁺ ICOS⁺ cells (r=0.948). The pSyk⁺ cells show both Tfh markers. Bcl6 expressing cells in this population produce IL21 or IFN-γ. Expression of Bcl6 RNA transcripts are observed in qRT-PCR analysis.

FIG. 4D

Purified CD4⁺ T cells activated in vitro with ICs exhibit Bcl6 expression and are pSyk⁺. These cells show subpopulation that produce IL-21 and IFN-γ or IL-21 and IL-17A. IFN-γ and IL-17A procuring cells with IL-21 production are different in total percentage of cells.

FIG. 4E

Treatment of purified CD4⁺ T cells with ICs, generate pSyk⁺Bcl6⁺ cells that produce IFN-γ or IL-17A along with IL-21. A modest correlation exists in these two populations (r=0.620).

FIG. 4F

Syk inhibitor P505 (10 nM), inhibits IL-21 production (shaded) in cells that are producing IL-21 from IC treatment (open) (A). Blocking of pSyk is also observed in similar experiment (B).

FIG. 5 establishes that FcR signaling and TLR9 signaling using their respective ligands promotes development of Tfh cells that express Bcl6 and produce IL-21 or IL-17A.

FIG. 5A

Combined treatment with ICs and CpG ODN 2006 of purified human CD4⁺ T cells enhances the expression of Bcl6-IL-21, and Bcl6-IL-17A compared to control population treated with CpG ODN 2006 alone.

FIG. 5B

Joint treatment with ICs and CpG ODN 2006 of naïve CD4⁺ T cells shows enhanced Bcl6⁺-IL-21⁺ and Bcl6⁺-IL-17A⁺ populations (control in left vs. treated in right).

FIG. 5C

Analysis of percentage increase in IL-21⁺Bcl6⁺ and IL17A⁺Bcl6⁺ cells from in vitro activation from various combination of CpG ODN and ICs and individual treatments.

FIG. 6 establishes that the FcR signaling is a strong inducer of genes in several key biological pathways i.e. TCR signaling, MHC class II, NF-κB and Proteasome complex compared to CD28 signal.

FIG. 6

Comparison of RNA-seq transcripts levels upon FcR cosignaling compared to CD28 using STRING 10.0. Analysis show upregulation of several key biological pathway transcripts levels observed upon control CD28. Accession No.: GSE127664

FIG. 7 establishes that two key genes that associate with HIV-1 infection are overexpressed at RNA-transcript level upon FcR signaling compared to CD28 cosignaling.

FIG. 7

Two key genes, C—C motif Chemokine Receptor 5 (CCR5) and Granzyme A (GZMA) are overexpressed from FcR cosignaling compared to CD28 control in human CD4⁺ T cells. CCR5 is a coreceptor for macrophage-tropic virus including HIV. GZMA is a serine protease necessary for the lysis of target cells such as infected by viruses by cytotoxic T lymphocytes.

FIG. 8 establishes that Tfh cells generated from FcR signaling are capable of driving the differentiation of human B cells into globulin producing cells.

FIG. 8

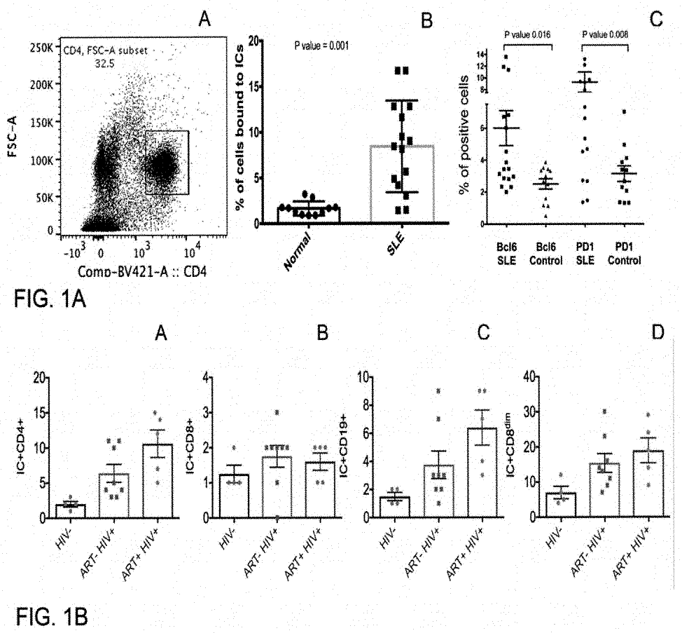

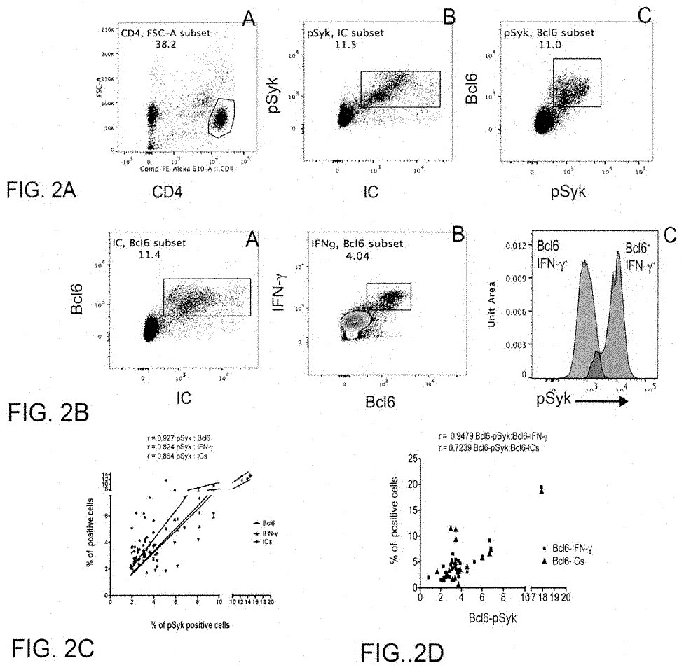

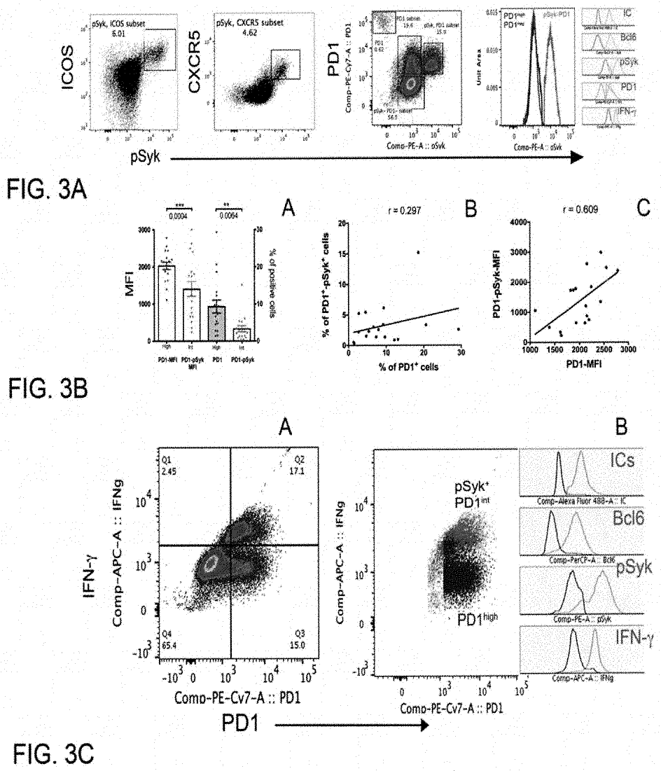

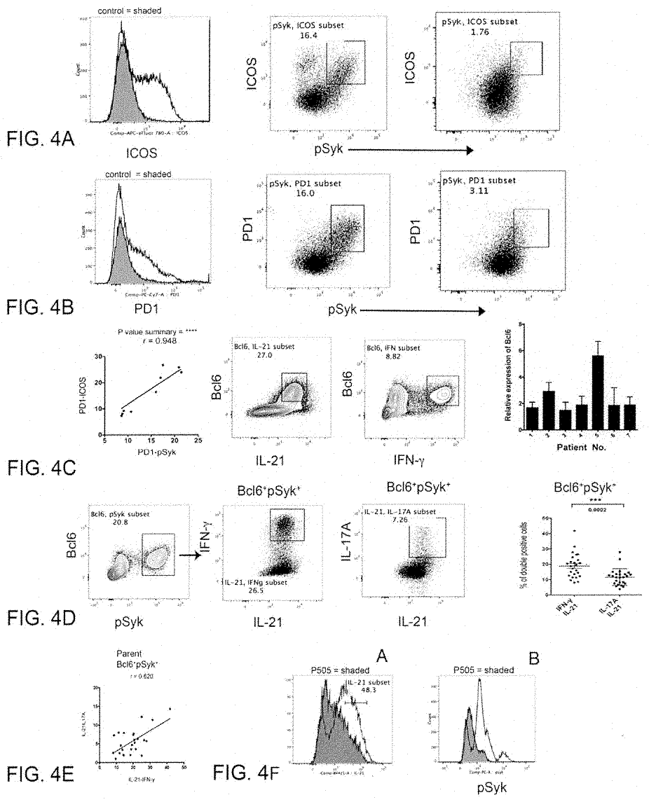

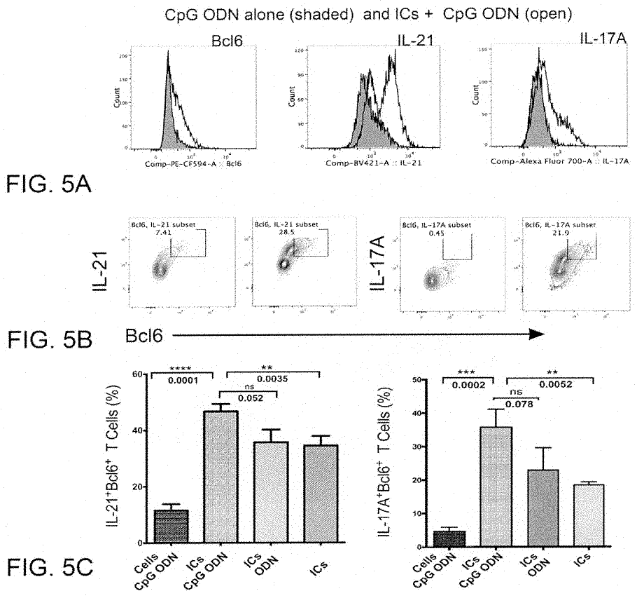

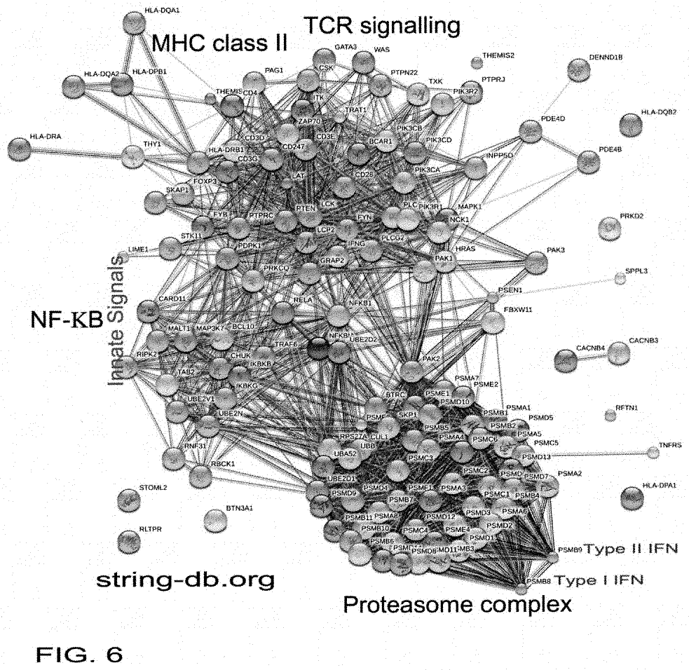

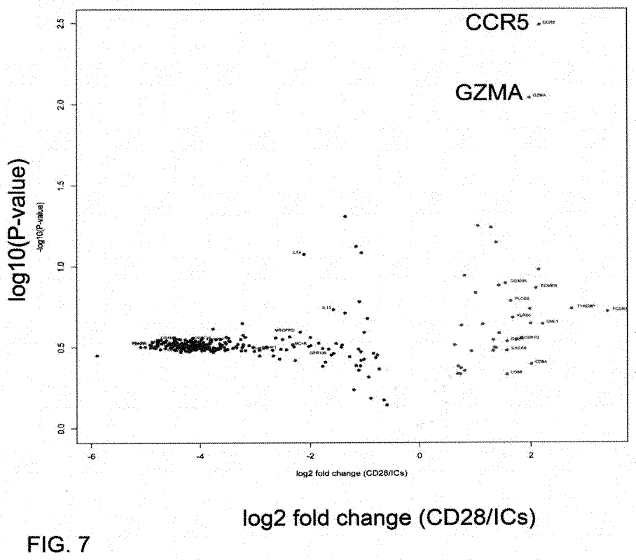

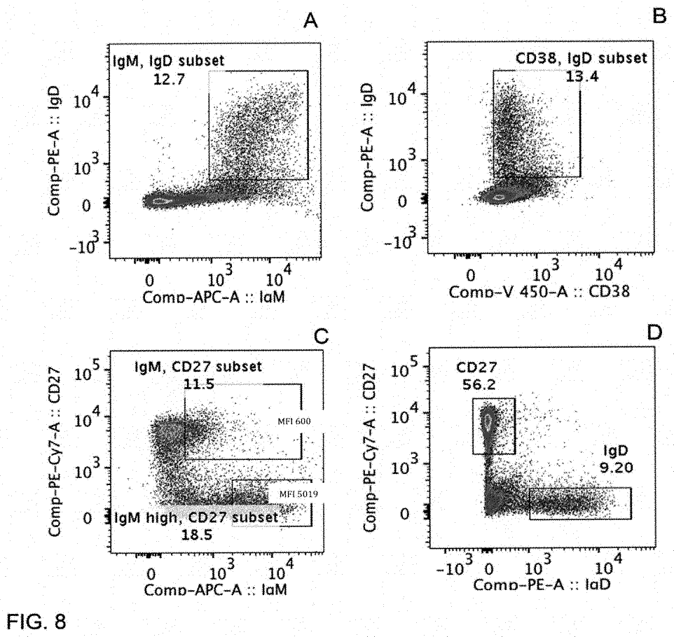

In vitro generated Tfh-like cells by IC treatment (FcR signal) upon co-culture with naïve human B cells successfully differentiate them leading to them to express IgM, IgD (A); CD38, IgD (B); CD27,IgM; and IgD in B cells (D).

What is claimed is:

1. A method where human naïve CD4⁺ T cells are converted to Tfh-like cells by activating them using ICs (purified from plasma/serum from disease condition opsonized with complement fragments) at a concentration of 0.1 μg to 10 μg or/and 0.1 μg to 10 μg of C5b-9 in the presence of anti-CD3 (0.1 to 1 μg coated) per one to ten million of purified naïve human CD4⁺ T cells. These Tfh-like cells express Fc receptor, as judged by the binding of Fluorochrome labeled ICs such as with Alexa Fluor 488 and these cells express Bcl6, a moderate amount of PD1, driven by pSyk⁺ signaling and produce both IFN-γ/IL-17A, and IL-21 (referred to as Tfh-like cells).

2. A process to differentiate human naïve CD4⁺ T cells to generate Tfh-like effector cells according to claim 1, where human naïve CD4⁺ T cells grown in the presence of appropriate combination of proinflammatory cytokines including but not limited to IL-2, IL-1β, IL-6, TGF-β, and IL-23 at various effective concentration of cytokines.

3. A method to screen blocking agents for inhibiting the signaling triggered by ICs ligation to Fc receptors on CD4⁺ T cells (pSyk signaling) that leads to the generation of CD4⁺ Tfh-like cells as claimed in 1 and 2 to ameliorate disease pathology.

4. Enumerate and correlate the amount of IC bound Tfh-like cells to monitor disease activity score such as SLEDAI in SLE and quantity of HIV-1 pro-viral nucleic acid positive CD4⁺ T cell population.

5. A process to develop screening assay for blocking the costimulatory signals triggered by ICs in naïve CD4⁺ T cells (pSyk⁺ signaling) for limiting the development of described Tfh-like cells that express PD1 and produce IL-21.

6. Development of a synthetic chemical, peptide, or a biological agent such as antibody, antibody drug conjugate to eliminate cells that express Fc receptors in CD4⁺ cell population and convert them to Tfh-like cells upon IC activation.

7. Use of the material developed in claim 6, to provide remission of disease associated symptoms and effects such as in autoimmunity and viral infections to provide relief from clinical symptoms of the disease.

8. Use of Tfh-like cells as generated in claim 3 for the development of plasma B cells from peripheral naïve/memory B cells using a co-culture system in vitro activated using purified ICs from the same patient.

9. Culture Tfh-like and naïve B cells activated with ICs as in 8 with addition and in the presence of appropriate cytokines/growth factors necessary to support B cell differentiation/growth and development of plasmablasts that produce immunoglobulins (autoantibodies).

10. Flow sort the plasma B cells that produce immunoglobulins. Use plasma B cells obtained as in claim 8 to generate cDNA libraries.

11. Use cDNA libraries prepared as in 10 for screening and identifying clones producing antibody protein against disease specific antigens.

12. Produce antibodies that are specific to disease associated antigens for prophylactic treatment of the disease pathology.

13. Test the blocking agents prepared in aim 6 to block the disease activity such as in autoimmune and viral pathology.

14. Test the blocking of agents as prepared in aim 6 to reduce expression of CCR5, a coreceptor on CD4⁺ T cells that facilitate the infections of macrophage-tropic virus including HIV-1.

15. To modulate the cytolytic activity of effector CD4⁺ T cells, these cells are prepared by activating with ICs purified from various disease pathologies and express Granzyme A.

16. Enhance the capability of the cells as in 14 to kill target cells that express antigens identified in ICs and/or viral infections to obtain a beneficial effect in the disease pathology.
